(19) United States
(12) Patent Application Publication
(10) Pub. No.: US 2018/0326024 A1
(43) Pub. Date: Nov. 15, 2018

(54) MATERIALS AND METHODS FOR TREATING EMBOLISM

(71) Applicant: University Hospital Ostrava, Ostrava-Poruba (CZ)

(72) Inventors: Vaclav Prochazka, Ostrava-Poruba (CZ); Jaromir Gumulec, Ostrava-Poruba (CZ); Jirka Macák, Ostrava-Poruba (CZ); Adéla Vrtková, Ostrava-Poruba (CZ)

(21) Appl. No.: 15/978,394

(22) Filed: May 14, 2018

Related U.S. Application Data
(60) Provisional application No. 62/505,658, filed on May 12, 2017.

(51) Int. Cl. A61K 38/48; A61K 9/00; A61P 7/02

(52) U.S. Cl. CPC A61K 38/4886; A61K 9/0019; A61K 9/19; C12Y 304/24087; A61P 7/02

(57) ABSTRACT

This document relates to materials and methods for treating and/or preventing an embolism (e.g., a cerebral embolism). For example, methods for using a composition including a disintegrin and metalloprotease with thrombospondin motif repeats (ADAMTS) polypeptide to treat a mammal having an embolism (e.g., a cerebral embolism) are provided.

### CROSS-REFERENCE TO RELATED APPLICATIONS

This application claims the benefit of Patent Application Ser. No. 62/505,658, filed on May 12, 2017. The disclosure of the prior application is considered part of (and is incorporated by reference in) the disclosure of this application.

### BACKGROUND

### 1. Technical Field

This document relates to materials and methods for treating and/or preventing embolism (e.g., a cerebral embolism). For example, this document provides methods for using a composition including one or more disintegrin and metalloprotease with thrombospondin motif repeats (ADAMTS) metalloproteases to treat a mammal having an embolism (e.g., a cerebral embolism). In some cases, a composition including one or more ADAMTS metalloproteases can be used to treat a mammal having an embolus causing large vessel occlusion (LVO) of brain circulation.

### 2. Background Information

Nearly 800,000 people in the United States have a stroke every year. For nearly 130,000 people a year, the stroke is fatal, making stroke the fifth most common cause of death in the United States (Centers for Disease Control and Prevention, Stroke Statistics). The cost of health care services, medicines to treat stroke, and missed days of work due to stroke costs the United States an estimated $33 billion each year (Mozzalifrian et al., Circulation, 133:e38-360 (2016)).

### SUMMARY

This document provides materials and methods for treating and/or reducing the risk of having an embolism (e.g., a cerebral embolism). For example, this document provides methods for using a composition including one or more ADAMTS (e.g., ADAMTS13) metalloproteases to treat a mammal having an embolism (e.g., a cerebral embolism) or at risk of experiencing an embolism (e.g., a cerebral embolism). In some cases, a composition including one or more ADAMTS metalloproteases can be used treat a mammal having an embolus causing LVO of brain circulation.

As demonstrated herein, low levels of ADAMTS13 metalloprotease activity contribute to vWF adhesion to the damaged endothelial cells and forming a large vWF multimers and thrombi. Having the ability to reduce the size and/or stickiness of an embolus ADAMTS13 provides a unique and unrealized opportunity to treat an embolism or to reduce the risk of having an embolism.

In general, this document features methods for treating a cerebral embolism in a mammal. In one aspect, a method for treating cerebral embolism in a mammal can include, or consist essentially of, administering a composition comprising an ADAMTS13 metalloprotease to a mammal identified as having an embolism, where the embolism includes a cardiac thromboembolus in a cerebral artery, and where one or more symptoms of ischemic stroke can be reduced.

The one or more symptoms can include slurring, confusion, difficulty understanding speech, paralysis, numbness, blurred vision, blackened vision, seeing double, headache, stumbling, dizziness, loss of balance, and/or loss of coordination. The cerebral artery can be the internal carotid artery, anterior cerebral artery, the middle cerebral artery, the posterior cerebral artery, vertebral artery, or the basilar artery.

The cardiac thromboembolus can comprise of red blood cells, fibrin, von Willebrand factor and/or vWF multimers, platelets, and/or inflammatory cells (e.g., neutrophils and monocytes) and plasma. The mammal can be a human.

A composition provided herein (e.g., a therapeutic composition containing one or more ADAMTS13 metalloproteases) can be administered by injection (e.g., an intra-arterial or intravenous). For example, an intra-arterial injection can be used to administer a composition provided herein into the internal carotid artery, anterior cerebral artery, the middle cerebral artery, the vertebral artery, the basilar artery, and/or the posterior cerebral artery by the local or loco-regional application.

A composition provided herein can include about 100 Units to about 300 Units of an ADAMTS13 metalloprotease per kg body weight of the mammal (e.g., about 200 Units of an ADAMTS13 metalloprotease per kg body weight of the mammal). A composition provided herein can include about 10,000 Units to about 15,000 Units of an ADAMTS13 metalloprotease (e.g., about 12.000 Units of an ADAMTS13 metalloprotease).

A method provided herein also can include one or more antithrombotic treatments (e.g., treatment with aspirin, warfarin, heparin, thrombin inhibitors, direct Xa inhibitors, thrombolytic treatment agents, streptokinase, urokinase, anistreplase, alteplase, reteplase, tenecteplase, staphylokinase, DNase, irreversible cyclooxygenase inhibitors, adenosine diphosphate receptor inhibitors, phosphodiesterase inhibitors, protease-activated receptor-1 antagonists, glycoprotein IIB/IIIA inhibitors, adenosine reuptake inhibitors, and/or thromboxane inhibitors). In some cases, mechanical thrombectomy, aspiration embolectomy, catheter-guided thrombolysis, percutaneous cerebral angioplasty, and/or stenting can be performed.

In another aspect, a method for treating cerebral embolism in a mammal can include, or consist essentially of, administering a composition comprising an ADAMTS13 metalloprotease to a mammal identified as having an embolism, where the embolism includes cardiac thromboembolus in a cerebral artery, where the cardiac thromboembolus is greater than 8 mm in size (LVO), and where the cardiac thromboembolus is reduced in size to less than 2 mm. In some cases, the cardiac thromboembolus can be eliminated from the cerebral artery. The cerebral artery can be the anterior cerebral artery, the middle cerebral artery, the posterior cerebral artery, vertebral artery, or the basilar artery.

In another aspect, a method for treating cerebral embolism, in a mammal can include, or consist essentially of, administering a composition comprising an ADAMTS13 metalloprotease to a mammal identified as laving an embolism, where the embolism includes a cardiac thromboembolus in a cerebral artery, and where the cardiac thromboembolus is augmented in adhesiveness and stickiness and resistant to thrombolysis.

The cardiac thromboembolus augmented in adhesiveness and stickiness can include an enhanced amount of von Willebrand factor (vWF). The enhanced amount of vWF can be at the thromboembolus surface. The vWF at the thromboembolus surface can be present in vWF/multimers. The cerebral artery can be the internal carotid artery, anterior cerebral artery, the middle cerebral artery, the posterior cerebral artery, vertebral artery, or the basilar artery.

The cardiac thromboembolus can compose of red blood cells, fibrin, von Willebrand factor/multimers, platelets, and/or inflammatory cells and plasma.

The mammal can be a human. The composition can be administered by injection (e.g., an intra-arterial or intravenous injection). For example, an intra-arterial injection can be into one or more of the internal carotid artery, anterior cerebral artery, the middle cerebral artery, the vertebral artery, the basilar artery, and the posterior cerebral artery. The composition can include about 100 Units to about 300 Units of an. ADAMTS13 metalloprotease per kg body weight of the mammal (e.g., about 200 Units of an ADAMTS 3 metalloprotease per kg body weight of the mammal). The composition can include about 10,000 Units to about 15,000 Units of an ADAMTS13 metalloprotease (e.g., about 2,000 Units of an ADAMTS13 metalloprotease). The method also can include one or more antithrombotic treatments (e.g., treatment with aspirin, warfarin, vitamin k antagonists, heparin, thrombin inhibitors, direct Xa inhibitors, streptokinase, urokinase, anistreplase, alteplase, reteplase, tenecteplase, staphylokinase, DNase, irreversible cyclooxygenase inhibitors, adenosine diphosphate receptor inhibitors, phosphodiesterase inhibitors, protease-activated receptor -1 antagonists, glycoprotein IIB/IIIA inhibitors, adenosine reuptake inhibitors, and/or thromboxane inhibitors). In some cases, mechanical thrombectomy, aspiration embolectomy,catheter-guided thrombolysis, percutaneous cerebral angioplasty, and/or stenting can be performed.

In less otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patients, and other reference mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1C contain images visualizing blood flow in the brain. FIG. 1A shows an angiography contrast image of an occlusion of the left M1-MCA origin with the severe left hemisphere hypo-perfusion. FIG. 1B shows an angiography contrast image of a DSA-follow up after mechanical thrombectomy procedure with pReset stent-retriever resulting in full perfusion recovery of the left MCA circulation territory; the lenticulostriates were well perfused with no distal embolization. FIG. 1C shows final perfusion image after left MCA complete recanalisation with GE-AngioViz.

FIGS. 2A-2G contain photographs of emboli retrieved from the brain circulation. FIG. 2A shows an embolus captured in a pREset Stent-retriever. FIG. 2B shows an embolus captured in an ACE64 Penumbra aspiration catheter. FIG. 2C shows fragmented emboli retrieved from brain circulation. These red thrombi with fibrous tissue were of cardiac origin in a patient with artrial fibrillation. FIG. 2D shows a single embolus with the fibrous tissue and the proximal fixation part not fragmented during the extraction procedure. FIG. 2E shows a yellow fibrous tissue thrombus completely extracted from circulation. This thrombus was of cardiac origin in a patient with atrial fibrillation. FIG. 2F shows a single embolus from an athero-thrombotic stroke (top), and a microscopic image of that embolus (bottom) showing that thrombotic material includes spheric red blood cells without compression of the thrombus and without neutrophils, with enough plasma surrounded red blood cells. FIG. 2G shows a single embolus from a cardio-embolic stroke (top), and a microscopic image of that embolus (bottom) showing that cardio-embolic material includes compressed red blood cells, less plasma content, lot of neutrophils (CD3 1) and monocytes (CD15). FIG. 2H is a Transmition Electron Microscopy image of a cardio-embolic thrombi extracted from the brain vessel showing compressed red blood cells with fibrin tissue and contracted thrombi.

FIGS. 3A-3D contain microscopic photographs of extracted emboli stained with various factors. FIG. 3A and FIG. 3B each represent an individual embolus stained with H&E, vWF, Carstairs, or CD15, FIG. 3C and FIG. 3D each represent an individual embolus stained with H&E, Carstairs, CD31 , vWF, or CD15. Red blood cells with granulocytes and CD15-activated myeloid cells with monocyte infiltration was frequently observed. Emboli surface and internal compartments are heavily covered by vWF multimers, with platelets adhered in the conglomerates.

FIG. 4 contains box plots comparing peripheral blood coagulation parameters of patients with less and more severe clinical status. A significant difference was observed in vWF levels at the time of admission to the hospital. Significantly higher levels were observed in those patients with NIHSS>15 at the time of admission.

FIG. 5 contains box plots comparing peripheral blood coagulation parameters with reference to patient's condition modified Ranklin Scale—(mRS) at 3 months (3M) after the stroke. A significant difference was observed in vWF levels. Significantly higher levels of vWF were observed in those patients with mRS>2 after three months.

FIG. 6A contains box plots showing the blood inflammatory response was activated significantly in patients at the Day 5 (+/−2). FIG. 6B contains box plots showing lymphocyte and monocyte significant activation in patients at Day 5 (+/−2).

FIGS. 7A and 7B are graphs of Spearman's rank correlation coefficient between selected variables in patients with acute ischemic stroke. Significant coefficients are circled. Correlation coefficients close to 1 (−1) suggest strong positive (negative) relationship between variables. Figures shows correlations between hemocoagulation, immune response, histology, and clinical outcome. FIG. 7A shows significant correlations between hemocoagulation analysis and histology, and between von Willebrand factor (vWF) and thrombotic parameters: platelets, vWF, and fibrin levels. These parameters are significantly correlated with each other. FIG. 7B reveals that histology and the inflammatory response shows a significant correlation with fibrin thrombus and inflammatory response parameters. Significant correlation coefficients are circled (level of significance 0.05).

FIG. 8 is a schematic showing an exemplary in vitro thrombolysis test.

FIG. 9 contains graphs showing age comparisons of clinical outcome. Age by three-month modified Rankin Scale score Multiple boxplots show whether patients with better three-month modified Rankin Scale score (3M-mRS) were younger or older than patients with worse three-month modified Rankin Scale score. There was a significant difference in terms of the age of all patients with reference to the three-month modified Rankin Scale score (P=0.007).

FIGS. 10A-10C show patient outcome following mechanical thrombectomy. FIG. 10A shows patient outcome following extraction devices and three-month modified Rankin Scale (3M-mRS) score: three months after the procedure, no significant difference was observed between the extraction devices used (P=0.98). FIG. 10B shows patient outcome following intravenous thrombolysis (IVT): no significant difference in the three-month modified Rankin Scale score (P=0.459) between the groups of patients with and without intravenous thrombolysis before the interventional procedure. FIG. 10C shows patient outcome following Treatment in Cerebral Isehemia (TICI) score for recanalization: significantly better clinical outcomes (P=0.02) were observed in patients with IRA score of 2-3 for recanalization of the large vessel occlusion (LVO) than in patients with a TICI score of 0-1 for recanalization.

FIG. 11. Scanning optical microscopy of the extracted cardiac-thrombi. Thrombi surface is densely packed with vWF in conjunction with inflammatory cells (Neutrophils and Monocytes).

### DETAILED DESCRIPTIONS

This document provides materials and methods for treating and/or reducing the risk of having an embolism (e.g., a cerebral embolism). In some cases, a composition including one or more ADAMTS (e.g., ADAMTS13) metalloproteases can be used to treat a mammal having an embolism (e.g., a cerebral embolism) or at risk of developing an embolism (e.g., a cerebral embolism). For example, a composition including one or more ADAMTS13 metalloproteases can be effective to reduce one or more symptoms associated with an embolism and/or can be effective to reduce the size and/or stickiness of an embolus (e.g., a lodged embolus). In some cases, a composition including one or more ADAMTS13 metalloproteases can be effective to prevent an embolus from lodging in a cerebral blood vessel (e.g., a cerebral artery). For example, a composition including one or more ADAMTS13 metalloproteases can be effective to reduce the size and/or stickiness of an embolus (e.g., a free embolus).

An embolus can be any unattached mass that travels through the bloodstream and is capable of creating an arterial occlusion (e.g., an embolism) at a site distant from its point of origin. An embolus lodging in a cerebral artery (e.g., a cerebral embolism) will most likely cause a stroke due to ischemia.

An embolus can include various substances. Examples of substances that can make up an embolus, include, without limitation, blood clots (e.g., a blood clot containing red blood cells, fibrin, vWF/multimers, platelets, and/or inflammatory cells (granulocytes, monocytes, eosinophils, and macrophages); a thromboembolus), cholesterol plaques and/or crystals; a cholesterol embolus), fat (e.g., globules and/or droplets; a fit embolus), gas (e.g., air; a gas embolus), pus (e.g., bacteria-containing pus; a septic embolus); tissue (e.g., fibrous tissue; a tissue embolus), foreign materials (e.g., talc and other small objects: a foreign body embolus), any debris that enters a pregnant mother's bloodstream (e.g., amniotic fluid and or fetal cells: an amniotic embolus), or tumor cells (e.g., myxoma tumor cells; a tumorous embolus). In some cases, an embolus can include one or more (e.g., two, three, four, five, or more) different substances. For example, an embolus can be a thromboembolus and/or a fibrous tissue embolus.

An embolus can be from a particular point of origin. Examples of points or origin include, without limitation, the heart (a cardiac embolus), an artery (an arterial embolus from aortic arch and/or carotid arteries), and a vein (a venous embolus such as a pulmonary embolism). In some cases, an embolus can be a cardiac origin embolus. The point of origin of an embolus can be determined using any appropriate method. For example, when an embolus is a thromboembolus, the thrombus from which the thromboembolus broke free from can be located and used to determine the point of origin of the thromboembolus. In some cases, imaging techniques (e.g., computerized tomography (CT) scanning such as CT angiography, magnetic resonance imaging (MRI) such as magnetic resonance angiography or magnetic resonance venography, carotid ultrasound, X-ray imaging such as cerebral angiography, digital subtracted angiography (DSA), transthoracal echocardipgraphy (TTE), and/or transesophageal echocardiography TEE)) can be used to locate the thrombus from which a thromboembolus broke free. In some cases, ultrasound, TTE, and/or TEE can be used to determine that the thromboembolus is a cardiac thromboembolus.

In some cases, the materials and methods provided herein can be used to reduce one or more symptoms of an embolism. Symptoms of cerebral embolism include, without limitation, trouble speaking (e.g., slurring), trouble understanding (e.g., confusion, difficulty understanding speech), paralysis or numbness (e.g., of the face, arm or leg), trouble seeing in one or both eyes (e.g., blurred vision, blackened vision, or seeing double), headache, and trouble walking (e.g., stumbling, dizziness, loss of balance, or loss of coordination). In some cases, a composition including one or mare ADAMTS13 metalloproteases can be used to eliminate one or more symptoms of an embolism.

In some cases, the materials and methods provided herein can be used to reduce the size of the embolus and/or fragment the embolus. The size of an embolus can be measured using any appropriate method. For example, the size of an embolus can be measured as the diameter of the embolus, the smallest distance across the embolus, and/or the length (e.g., total length) of the embolus. In some cases, the length of an embolism can be determined using, for example, CT angiography and/or DSA. In some cases, a composition including one or more ADAMTS metalloproteases can be used to reduce the size of an embolus large enough to cause an embolism (e.g., a cerebral embolism). For example, a composition including one or more ADAMTS metalloproteases can be used to reduce the size of an embolus greater than about 8 mm in size (e.g., greater than about 7.8 mm, greater than about 7.5 mm, greater than about 7.3 mm, greater than about 7 mm, or greater than about 6.6 mm in total length). In some cases, an embolus can be reduced in size to be less than about 3 mm in size (e.g., less than about 2.9 mm, less than about 2.8 mm, less than about 2.7 mm, less than about 2.5 mm, less than about 2.2 mm, less than about 2 mm, less than about 1.8 mm, less than about 1.5 mm, less than about 1.3 mm, less than about 1.0 mm, less than about 0.8 mm, less than about 0.5 mm, less than about 0.3 mm, or less than about 0.1 mm size). In some cases, the materials and methods provided herein can be used to eliminate the embolus. In some cases, the size of the embolus can be reduced in size by about 10% to about 100% (e.g., about 20%, about 30%, about 40%, about 50%, about 60%, about 70%, about 80%, or about 90%) of volume. For example, a composition including one or more ADAMTS13 metalloproteases can be used to reduce the size of a lodged embolus to increase blood flow and treat an arterial occlusion. For example, a composition including one or more ADAMTS13 metalloproteases can be used to reduce the size of a free embolus to prevent it front lodging in an artery and causing an arterial occlusion.

In some cases, the materials and methods provided herein can be used to reduce the stickiness of an embolus. As used herein, the “stickiness” of an embolus refers to the ability of the embolus to adhere to an artery (e.g., a cerebral artery) endothelial cells. In some cases, a sticky embolus can be a thromboembolus containing red blood cells, fibrin, vWF/multimers, platelets, and/or inflammatory cells (granulocytes, monocytes, eosinophils, and macrophages). For example, a composition including one or more ADAMTS13 metalloproteases can be used to reduce the stickiness of a lodged embolus (e.g., by reducing the amount of the large vWF/multimers at the thrombus surface) to dislodge the embolus and treat an arterial occlusion and protect endothelial cells. In some cases, the amount of vWF at the thrombus surface can be reduced by about 10% to about 100% (e.g., about 20%, about 30%, about 40%, about 50%, about 60%, about 70%, about 80%, or about 90%). For example, a composition including one or more ADAMTS13 metalloproteases can be used to reduce the stickiness of a free embolus to prevent it from lodging in an artery and causing an arterial occlusion.

When treating and/or preventing an embolism as described herein, the embolism can be in any particular location. Examples of locations in which an embolus can lodge to create a LVO (e.g., an arterial occlusion) include, without limitation, the brain (e.g., a cerebral artery such as the internal carotid artery, anterior cerebral artery (ACA), the middle cerebral artery (MCA), the posterior cerebral artery (PCA), vertebral artery (VA), and the basilar artery (BA). In some cases, the embolism treated as described herein can be a cerebral embolism.

Any type of mammal having an embolism or at risk for developing an embolism can be treated as described herein. For example, humans and other primates such as monkeys having an embolism or at risk of developing an embolism can be treated with a composition including one or more ADAMTS (e.g., ADAMTS13) metalloproteases. In some cases, dogs, cats, horses, cows, pigs, sheep, rabbits, mice, and rats can be treated with a composition including one or more ADAMTS (e.g., ADAMTS13) metalloproteases described herein.

Any appropriate method can be used to identify a mammal as having an embolism or at risk of developing an embolism. For example, physical examination (e.g., checking for blood pressure, atherosclerosis, and/or cholesterol crystals or clots in the blood vessels at the back of your eyes), blood tests (e.g., for blood clotting and/or blood sugar levels), imaging techniques (e.g., CT scanning such as CT angiography, MRI such as magnetic resonance angiography or magnetic resonance venography, carotid ultrasound, X-ray imaging such as cerebral angiography, electrocardiography (ECG of EKG) and/of echocardiogram) can be used to identify a human or other mammal as having an embolism or as being at risk of developing an embolism.

Once identified as having an embolism or as being at risk for developing an embolism, the mammal can be administered or instructed to self-administer one or more ADAMTS (e.g., ADAMTS13) metalloproteases (e.g., a composition including one or more ADAMTS13 metalloproteases). In some cases, a mammal being at risk for developing an embolism can have one or more conditions associated with the formation and/or presence of one or more emboli (e.g., cardiac thromboemboli). Examples of conditions associated with the formation and/or presence of one or more cardiac thromboemboli include, without limitation, acute myocardial infarction (AMI) and atrial fibrillation (AF).

A composition including one or more ADAMTS e.g., ADAMTS13) metalloproteases provided herein can be administered using any appropriate technique. Examples of techniques that can be used to administer a composition including one or more ADAMTS13 metalloproteases include, without limitation, injection (e.g., intra-arterial or intravenous injection). For example, a composition including one or more ADAMTS13 metalloproteases can be intra-arterially injected (e.g., through the diagnostic catheter or microcatheter) into one or more of the internal carotid artery (ICA), ACA, the MCA, the PCA, the VA, and the BA. In some cases, a composition including one or more. ADAMTS13 metalloproteases can be administered locally or systemically. For example, a composition including one or more ADAMTS13 metalloproteases can be administered systemically.

A composition including one or more ADAMTS (e.g., ADAMTS13) metalloproteases provided herein can include any appropriate ADAMTS polypeptide. In some cases, an ADAMTS metalloproteases can cleave vWF. In some cases, an ADAMTS metalloprotease can have thrombospondin motif. In some cases, an ADAMTS13 metalloprotease can be a recombinant ADAMTS13 metalloprotease. In some cases, an ADAMTS13 metalloprotease can be a synthetic ADAMTS13 polypeptide. Examples of human ADAMTS metalloproteases include, without limitation, ADAMTS1, ADAMTS2, ADAMTS3, ADAMTS4, ADAMTS5 (ADAMTS11) ADAMTS6, ADAMTS7, ADAMTS8 (METH-2) ADAMTS9, ADAMTS10, ADAMTS12, ADAMTS13, ADAMTS14, ADAMTS15, ADAMTS16, ADAMTS17, ADAMTS18, ADAMTS19, and ADAMTS20. For example, an ADAMTS metalloprotease can be an ADAMTS13 metalloprotease. Examples of ADAMTS13 metalloprotease include, without limitation, the human ADAMTS13 metalloprotease having the amino acid sequence set forth in GenBank® accession Nos. AAQ88485 (e.g., Version AAQ88485.1), AAL11095 (e.g., Version AAL11095.1), NP_620596 (e.g., Version NP_620596.2) NP_620595 (e.g., Version NP_620595.1), NP_620594 (e.g., Version NP_620594.1) CAI12852 (e.g., Version CAI12852.1), CAI12851 (e.g., Version CAI12851.1), and CAI112850 (e.g., Version CAI12850.1). In some cases, an ADAMTS13 metalloprotease provided herein can be a fragment of an ADAMTS13 metalloprotease provided the fragment maintains the ability to cleave vWF. In some cases, an ADAMTS13 metalloprotease provided herein can be a variant sequence (e.g., can have at least 75 percent sequence identity (e.g., at least 80%, at least 82%, at least 85%, at least 88%, at least 90%, at least 93%, at least 95%, at least 97% or at least 99% sequence identity)) to an ADAMTS13 metalloprotease provided the polypeptide maintains the ability to cleave vWF. For example, an ADAMTS13 metalloprotease can have one or more deletions, additions, and/or substitutions of one or more amino acids provided the polypeptide maintains the ability to cleave vWF. The ability to cleave vWF can be assessed using any appropriate method. For example, polypeptide assays such as western blotting can be used to detect the size of vWF and/or vWF multimers to determine whether or not vWF has been cleaved.

A composition including one or more ADAMTS (e.g., ADAMTS13) metalloproteases provided herein can include any appropriate amount of ADAMTS metalloprotease. In some cases, a composition including one or more ADAMTS13 metalloproteases can contain from about 500 Units to about 20,000 Units of an ADAMTS13 metalloprotease (e.g., about 500 Units to about 10,000 Units, about 500 Units to about 5,000 Units, about 500 Units to about 1,000 Units, about 1,000 Units to about 20,000 Units, about 2,000 Units to about 20,000 Units, about 5,000 Units to about 20,000 Units, about 10,000 Units to about 20,000 Units, about 15,000 Units to about 20,090 Units, about 800 Units to about 15,000 Units, or about 1,000 Units to about 2,000 Units of an ADAMTS13 metalloprotease). For example, a composition formulated for administration to an adult human weighing about 60 kg can contain about 10,000 Units to about 15,000 Units (e.g., about 12,000 Units) of an ADAMTS13 metalloprotease.

A composition including one or more ADAMTS (e.g., ADAMTS13) metalloproteases provided herein can be administered to a mammal having an embolism (e.g., a cerebral embolism) or at risk of developing an embolism (e.g., a cerebral embolism) in a combination therapy with one or more additional agents/therapies used to treat an embolism and protect endothelial cells. For example, a combination therapy used to treat a mammal (e.g., a human) having a cerebral embolism associate with one or more thromboemboli (e.g., cardiac thromboemboli) can include administering to the mammal a composition including one or more ADAMTS13 metalloproteases and (a) one or more antithrombotic treatments (e.g., treatment with aspirin, warfarin, vitamin k antagonists, heparin, thrombin inhibitors, and/or direct Xa inhibitors), (b) treatment with one or more thrombolytic drugs (e.g., streptokinase, urokinase, end or anistreplase), (c) treatment with one or more tissue plasminogen activators (e.g., alteplase, reteplase, tenecteplase, and/or staphylokinase). (d) treatment with one or more antiplatelet drugs (e.g., irreversible cyclooxygenase inhibitors, adenosine diphosphate receptor inhibitors, phosphodiesterase inhibitors, protease-activated receptor-1 antagonists, glycoprotein IIB/IIIA inhibitors, adenosine reuptake inhibitors, and/or thromboxane inhibitors), (e) treatment with one or more agents that stimulate ADAMTS13 polypeptide production/secretion, (f) treatment with one or more agents that inhibit the degradation of ADAMTS13 metalloproteases, (g) treatment with one or more agents that enhance ADAMTS13 metalloprotease activity, and/or (h) treatment with one or more agents that inhibit ADAMTS13 metalloprotease clearance from circulation. In some cases, a combination therapy used to treat a mammal (e.g., a human) having a cerebral embolism associate with one or more thromboemboli (e.g., cardiac thromboemboli) can include administering to the mammal a composition including one or more ADAMTS13 metalloproteases in combination with any one or more of (a)-(h) of the preceding sentence in addition to performing (a) surgery, (b) mechanical clot retrieval (e.g., mechanical thrombectomy), (c) catheter-guided thrombolysis, (d) aspiration embolectomy (e.g., Penumbra and Solumbra techniques), (e) percutaneous cerebral angioplasty, and/or (f) stenting. In some cases, a combination therapy used to treat a mammal (e.g., a human) having a cerebral embolism associate with one or more thromboemboli (e.g., cardiac thromboemboli) can include administering to the mammal a composition including one or more ADAMTS13 metalloproteases and performing (a) surgery, (b) mechanical clot retrieval (e.g., mechanical thrombectomy), (c) catheter-guided thrombolysis, (d) aspiration embolectomy (e.g., Penumbra and Solumbra techniques), (e) percutaneous cerebral angloplasty, and/or (f) stenting.

In embodiments where one or more ADAMTS (e.g., ADAMTS13) metalloproteases are used in combination with one or more additional agents to treat a mammal having an embolism (e.g., a cerebral embolism) or at risk of developing an embolism (e.g., a cerebral embolism), the one or more additional agents can be administered at the same time or independently. For example, the composition including one or more ADAMTS13 metalloproteases can be administered first, and the one or more additional agents administered second, or vice versa. In embodiments where one or more ADAMTS (e.g., ADAMTS13) metalloproteases are used in combination with one or more additional therapies to treat a mammal having an embolism (e.g., a cerebral embolism) or at risk of developing an embolism (e.g., a cerebral embolism), the one or more additional therapies can be performed at the same time or independently of the administration of one or more ADAMTS13 metalloproteases. For example, a composition including one or more ADAMTS13 metalloprotease can be administered before, during, or after the one or more additional therapies are performed.

In some cases, one or more ADAMTS (e.g., ADAMTS13) metalloproteases can be formulated into a pharmaceutically acceptable composition for administration to a mammal having an embolism (e.g., a cerebral embolism) or at risk of developing an embolism (e.g., a cerebral embolism). For example, a therapeutically effective amount of ADAMTS13 metalloproteases can be formulated together with one or more pharmaceutically acceptable carriers (additives) and/or diluents. A pharmaceutical composition can be formulated for administration in solid or liquid form including, without limitation, sterile solutions, suspensions, sustained-release formulations, tablets, capsules, pills, powders, and granules.

Pharmaceutically acceptable carriers, tillers, and vehicles that can be used in a pharmaceutical composition described herein include, without limitation, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A pharmaceutical composition containing one or more ADAMTS (e.g., ADAMTS13) metalloproteases can be designed for oral, parenteral (including subcutaneous, intramuscular, intravenous, and intradermal), or inhaled administration. When being administered orally, a pharmaceutical composition containing one or more ADAMTS (e.g., ADAMTS13) metalloproteases can be in the form of a pill, tablet, or capsule. Compositions suitable for parenteral administration include aqueous and non-aqueous sterile injection solutions that can contain anti-oxidants, buffers, bacteriostats, and solutes which render the formulation isotonic with the blood of the intended recipient: and aqueous and non-aqueous sterile suspensions which may include suspending agents and thickening agents. Compositions for inhalation can be delivered using, for example, an inhaler, a nebulizer, and/or a dry powder inhaler. The formulations can be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules, and tablets.

Effective doses can vary depending on the size of the embolus and/or the severity of the embolism, risk of developing an embolism, the route of administration, the age and general health condition of the subject, excipient usage, the possibility of co-usage with other therapeutic treatments such as use of other agents, and the judgment of the treating physician.

An effective amount of a composition containing one or more ADAMTS (e.g., ADAMTS13) metalloproteases provided herein can be any amount that reduces the severity of one or more symptoms of embolism (e.g., slurring, confusion, difficulty understanding speech, paralysis, numbness, blurred vision, blackened vision, seeing double, headache, stumbling, dizziness, loss of balance, and/or loss of coordination) without producing significant toxicity to the mammal. In some cases, an effective amount of one or more ADAMTS13 metalloproteases can be from about 100 Units to about 300Units (e.g., about 200 Units) of an ADAMTS13 metalloprotease per kg body weight. For example, a composition containing one or more ADAMTS13 metalloproteases can be effective to deliver about 200 Units of an ADAMTS13 metalloprotease per kg body weight (e.g., for administration to a rabbit weighing about 2.5 kg to about 3 kg, a composition can include about 500 Units to about 600 Units of ADAMTS13 metalloproteases; and for administration to an adult human weighing about 60 kg, a composition can contain about 10,000 Units to about 15,000 Units (e.g., about 12,000 Units) of ADAMTS13 metalloprotease). The effective amount can remain constant or can be adjusted as a sliding scale or variable dose depending on the mammal's blood level of vWF/FVIII, decreased ADAMTS13 activity, and/or response to treatment. Various factors can influence the actual effective amount used for a particular application. For example, the frequency of administration, duration of treatment, use of multiple treatment agents, route of administration, and severity of the embolism may require an increase or decrease in the actual effective amount administered.

The frequency or administration can be any frequency that reduces the severity of a symptom of embolism without producing significant toxicity to the mammal. For example, the frequency of administration can be from about once a week to about three times a day, from about twice a month to about six times a day, or from about twice a week to about once a day. The frequency of administration can remain constant or can be variable during the duration of treatment. A course of treatment with a composition containing one or more ADAMTS (e.g., ADAMTS13) metalloproteases can include rest periods. For example, a composition containing one or more ADAMTS (e.g., ADAMTS13) metalloproteases can be administered daily over a two-week period followed by a two-week rest period, and such a regimen can be repeated multiple times. As with the effective amount, various factors can influence the actual frequency of administration used for a particular application. For example, the blood level of vWF/FVIII, decreased ADAMTS13 activity, decreased inflammatory response, the effective amount, duration of treatment, use of multiple treatment agents, route of administration, and severity of the embolism may require an increase or decrease in administration frequency.

An effective duration for administering a composition containing one or more ADAMTS (e.g., ADAMTS13) metalloproteases provided herein can be any duration that reduces the severity of a symptom of the embolism and/or prevents the development of an embolism without producing significant toxicity to the mammal. For example, the effective duration can vary from several days to several weeks, months, or years, in some cases, the effective duration for the treatment of an embolism and/or the prevention of the development of an embolism can range in duration from about one month to about 10 years. Multiple factors can influence the actual effective duration used for a particular treatment. For example, an effective duration can vary with the frequency of administration, effective amount, use of multiple treatment agents, route of administration, and severity of the condition being treated.

In certain instances, a course of treatment and the severity of one or more symptoms related to the embolism and/or indicators of risk of developing on embolism can be monitored. Any appropriate method can be used to determine whether or not the severity of a symptom is reduced. For example, the severity of a symptom of an embolism can be assessed using physical examination (e.g., neurological examination, NIHSS, mRS), blood tests (e.g., haemocoagulation, blood count, and/or inflammatory response), imaging techniques (e.g., CT scanning such as CT angiography, ASPECT score calculation, MRI such as magnetic resonance angiography, duplex Doppler ultrasound, X-ray imaging such as cerebral angiograph-DSA, ECG, and/or echocardiogram at different time points.

The invention will be further described in the following examples, which do not limit the scope of the invention described in the claims.

### EXAMPLES

### Example 1

### VWF, ADAMTS13, and Inflammatory Response

### Mechanical Thrombectomy

Mechanical thrombectomy is recently the method of choice for the large vessel occlusion (LVO) of the intracranial artery in acute ischemic stroke treatment with 85-90% technical success rate after failed IVT (intravenous thrombolysis). Stein retriever, penumbra aspiration, Solumbra combination, cerebral angioplasty and/or stenting are the techniques most often used for the intracranial thrombus extraction. Enclovascular procedure time, thrombolysis in cerebral infarction (TICI) recanalization grade, and risk factors (e.g., diabetes mellitus (DM), hypertension, and, arrhythmia) are the main factors influencing a 3M-mRS outcome after an ischemic stroke.

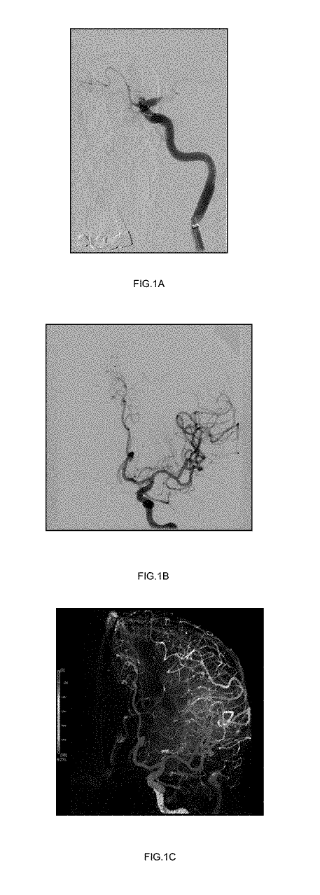

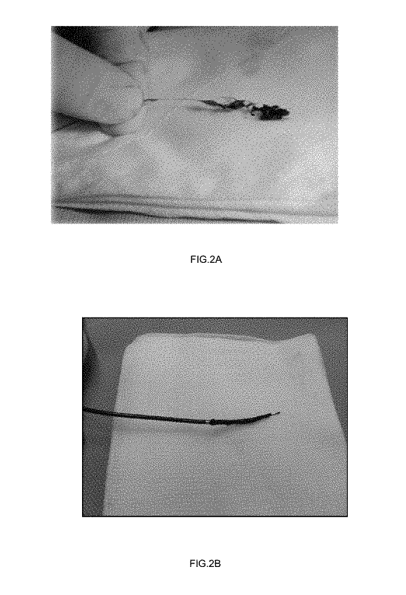

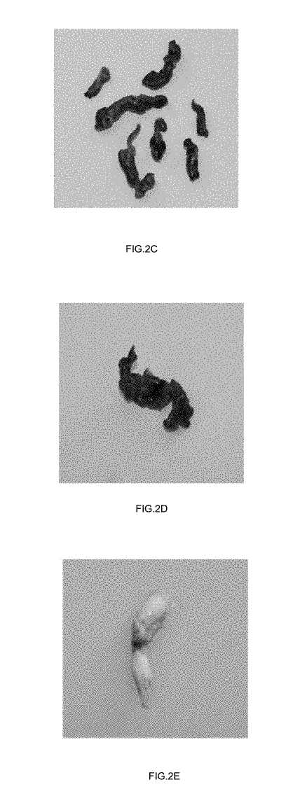

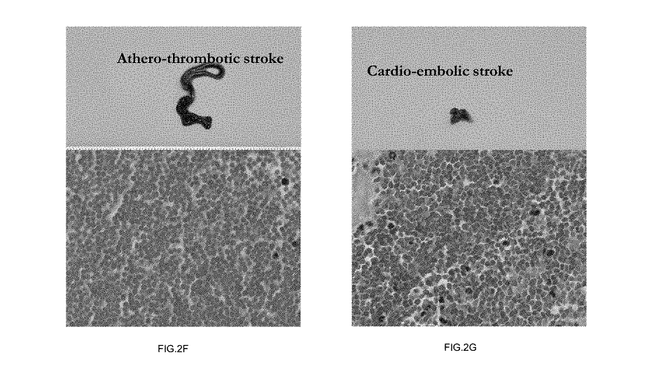

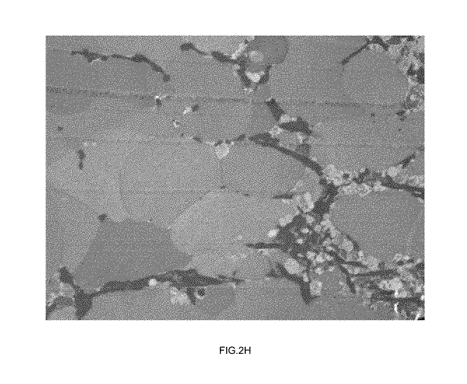

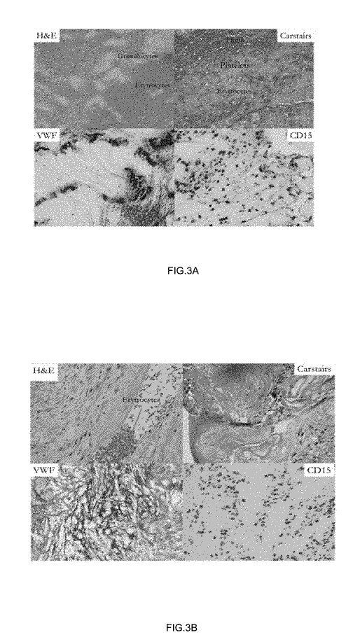

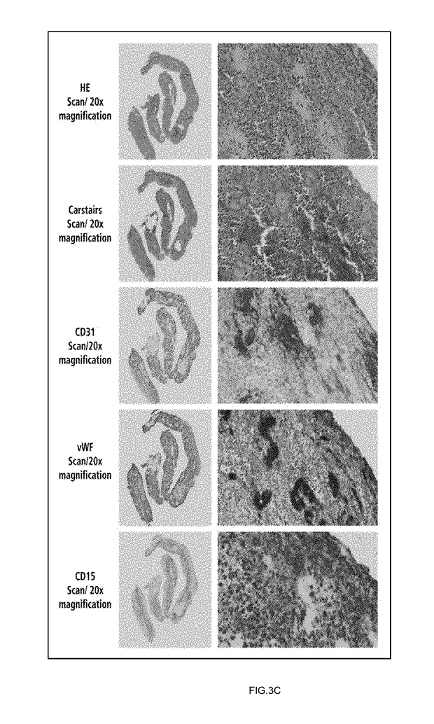

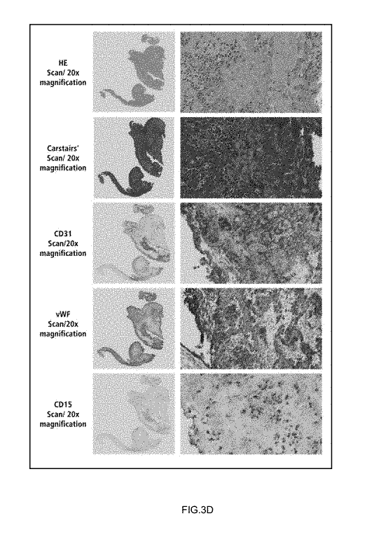

Procedure time of the sticky emboli extraction and the endothelial cells are influenced by the blood coagulation parameters especially platelets, fibrinogen, vWF/multimers. ADAMTS13 and the inflammatory cell respond (FIG. 1 and FIG. 2).

### Cardio-Embolic Thrombi

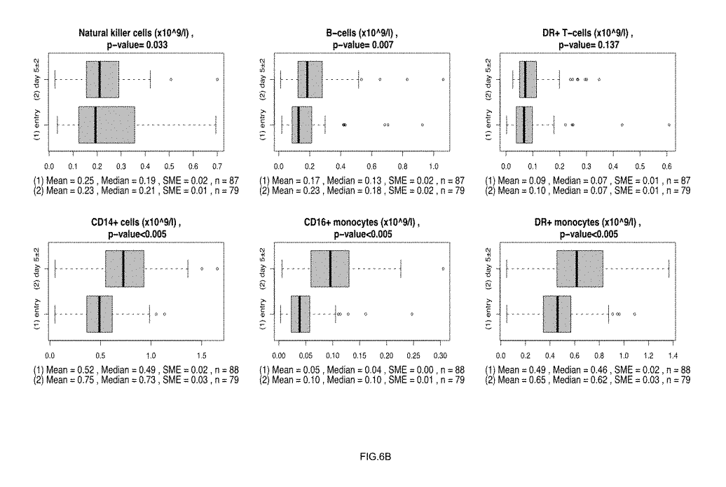

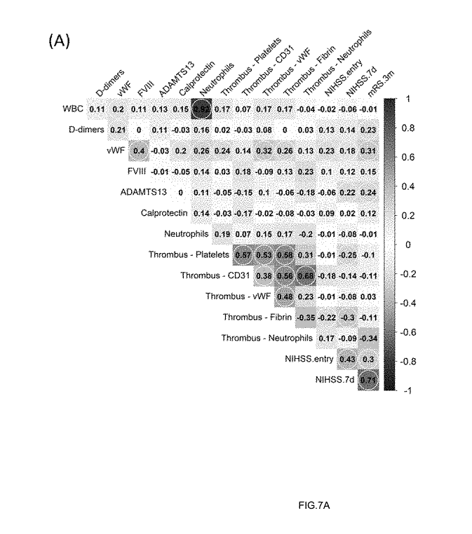

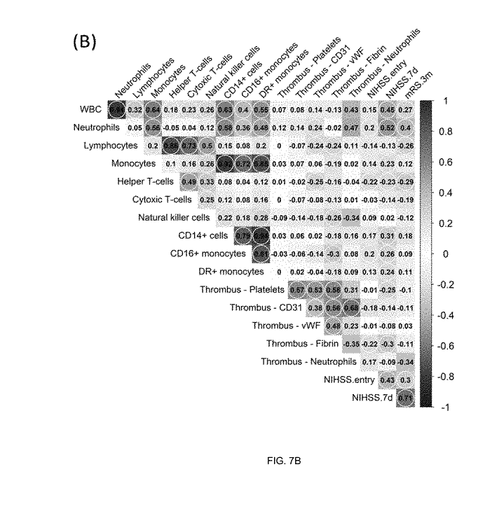

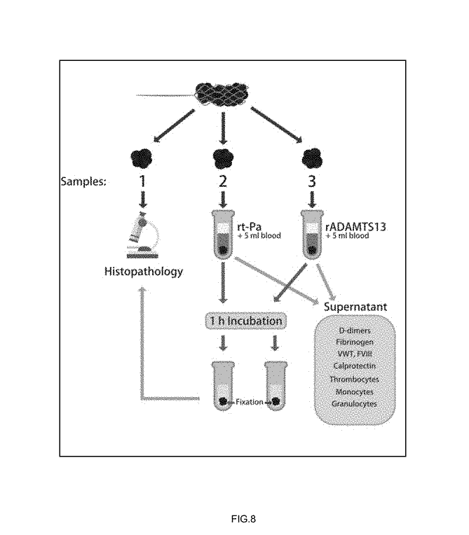

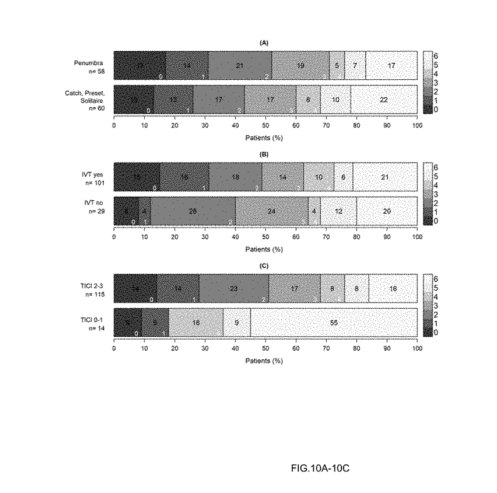

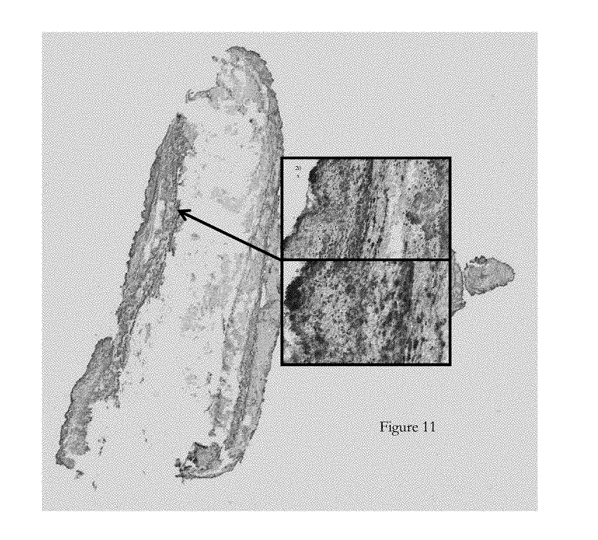

85 patients with acute ischemic stroke were analyzed. Emboli contained significant number of granulocytes, vWF, and platelets. Once, an embolus from cardiac origin wedged in a tiny brain artery, incomplete occlusion led to a significant acceleration of the blood flow around the thrombus. Acceleration of the blood flow increased the shear stress and activation of vWF and platelet aggregation. Reduced ADAMTS13 activity was found in these patients. Patients with high vWF, low ADAMTS13 activity, and higher vWF/ADAMTS13 ratio were found to have worse outcome (FIG. 4. FIG. 5, and FIG. 10). Neutrophil extracellular traps (NETs) and calprotectin were found to have a strong antibacterial impact. Calprotectin comprises up to 60% of cytoplasmic neutrophil content (FIG. 6 and FIG. 7).

### Histopathology

Thromboemboli were sorted into four groups (1. Red blood cells dominated (CR); 2. Approximately the same proportions of red cells and fibrin (plasma); 3. Fibrin predominance (plasma); and 4. Organized thromboemboli) as described elsewhere (see, e.g., Simon, et al., J. Neuroradial., 42:86-92 (2015)).

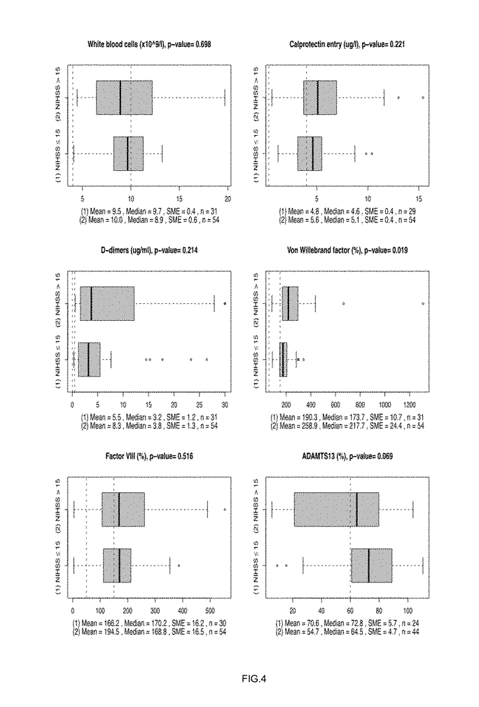

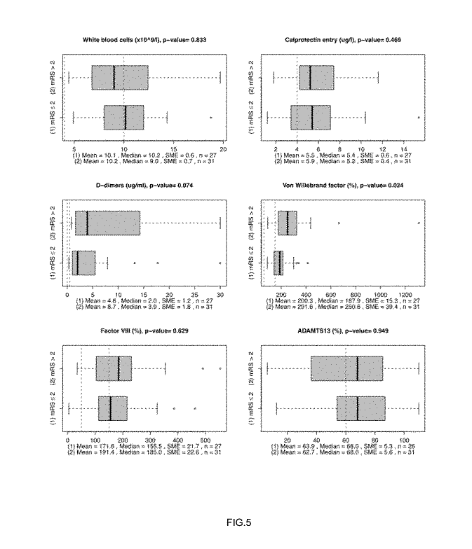

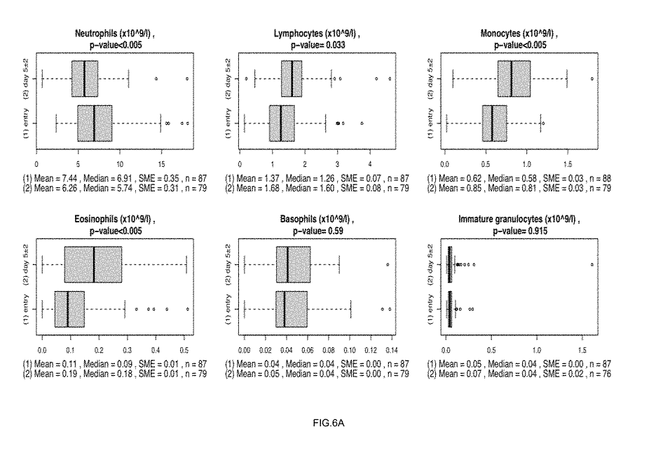

The relative ratio of the red blood cells and plasma in the samples were evaluated by hematoxylin-eosin staining. The measurement was performed on QuickPHOTO CAMERA. The amount of the vWF/FVIII was stained immunohistochemically and calculated. Cell analysis was made by immunohistology using CD31 and CD15 antibodies. CD31 is expressed in endothelium, on platelet surfaces, in myeloid cells, and in NK while CD15 is expressed in monocytes (FIGS. 3A-3D).

The number of platelets and fibrin was evaluated by Carstairs staining. The material was fixed with 4% buffered formalin, not exceeding 48 hours. The number of eosinophilic leukocytes in four large magnification fields was also evaluated.

The evaluation of the positive results took place after scanning digital photos and after processing QuickPHOTO CAMERA. This program allows accurate measurement of the area of positive results as a percentage of the total area of digital photography. Measurement were taken using 4× and 10× magnification lenses. One lens field of 4× affects a 20 mm² area, the 10× lens affects a size range of 3 mm². The measured values therefore corresponded to the percentages of the image area on the screen. For the 4× lens, the screen size of 2 mm² on the 10× lens was the size of the screen area of 0.3 mm². Five measurements from different sites were obtained from each thromboemboli.

These results demonstrated that thromboemboli with higher vWF/FVIII and lower ADAMTS13 activity are more sticky to the vessel wall of the brain arteries during the aspiration or extraction procedure, and in particular in conjunction with the rigid consistency prolongs the procedure time length during the mechanical thrombectomy.

### Example 2

### In Vitro Lysis of Brain Emboli

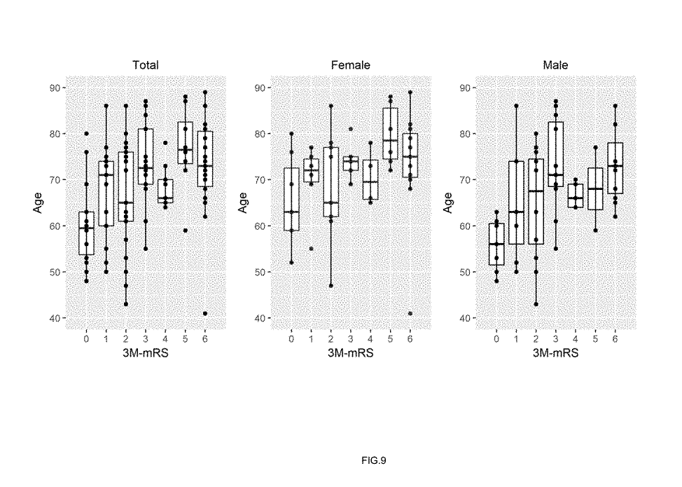

In Vitro lysis of brain emboli by ADAMTS13 is evaluated as shown in FIG. 9.

The thrombolytic effect of the recombinant ADAMTS13 metalloprotease (rADAMTS13) are investigated on the freshly extracted emboli from the brain vessels. Emboli are extracted during interventional procedures and are cut into three parts.

The first part is placed into a transport vial with fixation solution (4% buffered formalin), not to exceed 48 hours in the fixation solution. Histopathological analysis for red blood cells (CR), proportions of red cells and fibrin plasma, fibrin predominance (plasma), vWF/FVIII, myeloid cells infiltration, and organization of thromboemboli is done as described in Example 1.

The remaining two parts are placed into separate glass bottles. The same amount of the blood volume (5 cc) is added to each sample to maintain the plasminogen effect on the emboli for the rt-PA effect and to preserve the circulatory blood parameters. 10 mg of rt-PA is placed in the second bottle and rADAMTS13 (200 Units) is added to the third bottle. Both samples are photographically video recorded for 60 minutes (standard time for thrombolysis application). Any remaining volume of the emboli samples after thrombolysis is sent separately for advanced histopathology. After thrombi removal, the supernatant is analyzed for blood coagulation parameters (e.g., for D-Dimers, vWF, FVIII, fibrinogen, platelets, and inflammatory parameter liberated from the granulocytes (calprotectin)) as described in Example 1.

Results from the histopathology analysis of all three parts of the embolus are compared as well as the supernatant samples for the thrombolytic effect.

### Example 3

### ADAMTS13 and Cerebral Artery Thrombus Composition Patient Outcome

### Following Mechanical Thrombectomy for Acute Ischemic Stroke

This example investigated the role of von Willebrand factor (vWF), the vWF-cleaving protease, ADAMTS13, the composition of thrombus, and patient outcome following mechanical cerebral artery thrombectomy in patients with acute ischemic stroke in a cohort study including 131 patients with ischemic stroke (<6 hours) with or without intravenous thrombolysis. In patients with acute ischemic stroke, mechanical cerebral artery thrombectomy resulted in a good clinical outcome in 47% of cases, with and without intravenous thrombolysis therapy.

### Material and Methods

### Study Design

All patients provided written informed consent to participate in the study. A prospective cohort study included 131 consecutive patients with acute ischemic stroke, having occurred within <6 hours, with or without the use of intravenous thrombolysis using recombinant tissue plasminogen activator (rtPA) (Actilyse, Boehringer Ingelheim, Germany). Following a mechanical thrombectomy procedure all patients included in the study had confirmed large vessel thromboembolic occlusion, and their neurological clinical status was assessed with the National institutes of Health Stroke Scale (NIHSS) obtained on hospital admission, after 24 hours, at day 7, and finally, at three months, using the three-month modified Rankin Scale.

Time indicators of acute ischemic stroke and hospital admission included onset-to-door (OTD), door-to-needle (DTN), onset-to-needle (OTN), and onset-to-recanalization (OTR) were evaluated. Early ischemic changes and collateral circulation were assessed by the Alberta Stroke Program for Early CT Score (ASPECTS). Computed tomography angiography (CTA), or digital subtraction angiography (DSA), was used for localization of large cerebral vessel occlusion and measurement of thrombus length.

Neuro-interventional procedure data was obtained by using the Treatment in Cerebral Ischemia (TICI) score, number of mechanical attempts, type al procedure, and procedure duration. The thromboemboli extracted using mechanical thrombectomy were immediately placed into a transport vial containing the fixative, 10% formalin, and transferred to the laboratory for processing for light microscopy and immunohistochemistry. At the same time, 40 ml of peripheral blond was collected from each patient to examine blood levels of on Willebrand factor (vWF), the von Willebrand factor-cleaving protease, ADAMTS13, and other coagulation factors and inflammatory mediators, using routine clinical laboratory standards and techniques, prior to initiation of the mechanical interventional procedure and later on day 5±2 days from the onset of the acute ischemic stroke.

### Study Population

A total of 131 patients 69 men (53%) and 62 women (47%), with a median of age 71 years (IDR, 53-82 years) underwent mechanical thrombectomy for cerebral large vessel occlusion. Intravenous thrombolysis therapy was used in 101 (78%) patients, which tiled to result in vessel recanalization. The Alberta Stroke Program for Early CT Score (ASPECTS) findings were in the IDR of 6-10 points in 129 patients (99%), and the ASPECTS scoring IDR was 3-5 points in 69% of patients, with collateral circulation, analyzed from the initial CTA imaging findings. Time parameters were obtained for all patients; the median of the time from the stroke onset to the mechanical thrombectomy procedure was 160 minutes (IDR : 100-270 min), and the median of procedure duration was 45 minutes (IDR; 25-85 min). Demographic variables, risk factors, and procedure description variables are shown in Table 1.

TABLE 1
Patient demographics and medical history.

| Variable | Categories | n (%) | OR (95% CI)* | P* |
|---|---|---|---|---|
| Sex | Female | 62 (47) | 0.95 (0.44-2.05) | 0.999 |
| | Male | 69 (53) | | |
| Diabetes mellitus | No | 99 (79) | 2.62 (0.98-6.98) | 0.064 |
| | Yes | 27 (21) | | |
| Hyperlipidemia | No | 55 (44) | 0.58 (0.27-1.27) | 0.237 |
| | Yes | 71 (56) | | |
| Hypertension | No | 27 (21) | 3.33 (1.17-9.52) | 0.026 |
| | Yes | 99 (79) | | |
| Smoking | No | 101 (80) | 1.18 (0.46-2.99) | 0.815 |
| | Yes | 25 (20) | | |
| Alcohol abuse | No | 124 (98) | NA | NA |
| | Yes | 2 (2) | | |
| Arrhythmia | No | 75 (60) | 1.09 (0.50-2.37) | 0.846 |
| | Yes | 51 (40) | | |
| Previous TIA | No | 121 (96) | 0.87 (0.12-6.42) | 0.999 |
| | Yes | 5 (4) | | |
| Previous stroke | No | 106 (84) | 1.95 (0.67-5.68) | 0.300 |
| | Yes | 20 (16) | | |

| Variable | Units | Median (IDR) | P** |
|---|---|---|---|
| Age | Years | 71 (53-82) | <0.001 |
| Systolic BP | mmHg | 151 (120-184) | 0.100 |
| Diastolic BP | mmHg | 85 (70-107) | 0.471 |
| Glycemia | mmol/l | 6.8 (5.3-9.2) | 0.832 |
| Cholesterol | mmol/l | 4.4 (3.4-6.1) | 0.469 |
| BMI | kg/cm² | 27.7 (22.9-35.2) | 0.187 |
| Inception to IVT | min | 92 (62-152) | 0.022 |
| Inception to DSA | min | 160 (100-270) | 0.561 |
| Arrival to IVT | min | 25 (17-40) | 0.140 |
| Arrival to DSA | min | 58 (20-90) | 0.493 |
| Procedure duration | min | 45 (25-85) | 0.139 |
| Recanalization | min | 200 (130-330) | 0.432 |

*Odds ratios for achieving better clinical status were computed (where possible), together with 95% confidence intervals and p-values of Chi-square test of independence (or Fisher’s exact test where necessary);
**P-values of Mann-Whitney U test were computed to compare patients with three-month modified Rankin Scale (3 M-mRS) score of 0-2 and patients with a three-month modified Rankin Scale score of 3-6.
Intravenous thrombolysis (IVT); digital subtraction angiography (DSA).

### Mechanical Interventions

Mechanical thrombectomy with a stent retriever, Penumbra aspiration device, or combination techniques was performed. A GE-Innova IGS 630 biplane system (GE Healthcare, Buc, France) was used for brain and vascular imaging of mechanical intervention and post-procedure three-dimensional (3D) X-ray angiography reconstruction.

Large vessel occlusions were recanalized with a pRESET thrombectomy device (Phenox GmbH, Bochum, Germany), Catch retriever (Balt Extrusion, Montmorency, France), Solitaire stent retriever (Medtronic, Dublin, Ireland), or Penumbra aspiration system (Penumbra, Inc., Alameda, Calif., USA). Cerebral vessels were accessed using a Flexor 6-7 F/90 cm guiding sheath (Cook Medical, Bjaeverskov, Denmark) or Terumo Destination guiding catheter (Terumo Medical Corp, Elkton, Md., USA). Large vessel occlusions were traversed with the Prowler Select Plus microcatheter (Codman & Shurtleff Inc., Raynham, Mass., USA) over several types of 0.014″ microwires. For the thrombus aspiration technique, ACE64 and 3MAX Penumbra catheters were used with the original suction pump. The type of anesthesia was recorded during the procedure, intravenous analgosedation with a laryngeal mask, or general anesthesia.

### Analysis of Hemostasis

Blood samples were collected from all patients before commencing the mechanical endovascular procedure and before the use of intra-arterial administration of heparin. Blood samples were analyzed for complete blood count (CBC) and reticulocytes using a Sysmex XN 9000 (Sysmex Corporation, Norderstedt, Germany). The coagulation tests, prothrombin time (PTT), activated partial thromboplastin time (APTT), thrombin time (TT), fibrinogen, and Factor VIII (FVIII), were measured on a fully automated blood analyzer (Siemens CS-5100) with Dade Innovin for PT, Pathromtin SL for APTT, Thromboclotin for TT, Dade Thrombin Reagent for fibrinogen, and Coagulation Factor VIII Deficient Plasma for FVIII. Immunoassay methods for D-dimers and vWF were conducted using Innovance D-dimers and Siemens vWF/Ag (Siemens Healthcare Diagnostics, Erlangen, Germany). ADAMTS13 activity was determined in citrated double-spun, platelet-poor plasma using the commercial Technozym® ADAMTS13 chromogenic enzyme-linked immunosorbent assay (ELISA) method (Technoclone, Vienna, Austria), according to the manufacturer's instructions.

Blood and Serum Analysis

The following parameters were determined prior to the mechanical interventional procedure and 5±2 days after the procedure. White blood cell (WBC) count and a differential were performed on the Sysmex XN2000 analyzer with Sysmex diagnostics. Lymphocyte subpopulations in peripheral blood were analyzed using a Navios flow cytometer and Beckman-Coulter diagnostic antibody cocktail Tetrachrome 45/56/19/3 (Ref: 66070703), CD8 PC7 (Ref: 737661), CD4-Alexa Fluor 750 (Ref: A94682), CD16-RPE (Ref: A07766), CD3-FITC/anti-HLA-DR-PE (Ref A07737), CD45-PC5 (Ref: A07785), and anti-HLA-DR-PC7 (Ref: B49180). Serum calprotectin concentrations were determined using a CalproLab ELISA kit (Calpro AS).

### Histopathology

Thrombus material obtained during mechanical thrombus extraction was fixed 10% phosphate-buffered formalin. Formalin-fixed specimens were embedded in paraffin wax, sectioned at 4 μm thickness, and stained with hematoxylin and eosin (H&E). Primary antibodies against vWF and CD31 were used to identify neutrophils and monocytes; anti-CD15 was used to identify neutrophils, eosinophils, and some monocytes; and Carstairs' histochemical method was used to identify platelets and fibrin.

Histological examination was performed without knowledge of the clinical findings and was based on detection analysis of vWF, platelet and fibrin accumulation, as well as neutrophil and monocyte deposits and erythrocyte-rich areas. The percentages of platelets, vWF-stained areas, fibrin, and CD31-positive cells were quantitatively determined. Histological sections were photographed with an Olympus BX41 microscope with an attached QuickPHOTO CAMERA (PROMICRA, Prague, Czech Republic). The positive immunostained results were quantified digitally using scanning digital photomicroscopy and processing with QuickPHOTO CAMERA software as a percentage of the total area of the digital image. Objective images were evaluated at ×4 and ×10 objective magnification for the measurement of positively immunostained stained areas in square centimeters, with the average of five measurements from different sample areas within the sections.

### Statistical Analysis

For univariate description of variables, the median and the interdecile range (IDR) was used, because of the occurrence of several outliers that could have affected the analysis. For graphical representation, boxplots were used, which were useful for exploring the structure of selected variables and effective graphical comparison of several groups of interest. Depending on the nature of the data, the Mann-Whitney U test, paired sign test, or Kruskal-Wallis test were used to evaluate statistically significant differences between groups.

The assessment of relationships between two variables used computation of the Spearman's rank correlation coefficient, with correlograms used to display better the relationships evaluated (significance level 0.05). The odds ratio (OR), a widely used measure of the association between the presence of a risk factor and outcome, was used and the Chi-square test of independence or Fisher's exact test were used, where relevant.

### Results

### Study Population

Of the 131 patients included in the study, men and women did not differ in terms of age distribution (P=0.239), or three-month modified Rankin Scale score (P=0.706), but younger patients fared better on the three-month modified Rankin Scale score (P=0.007) (FIG. 11). The right hemisphere was involved in acute ischemic stroke in 49 patients, the left hemisphere in 63 patients, and the posterior cerebral circulation was involved in 17 patients. Hypertension was clinically diagnosed in 99 patients (79%), and patients without hypertension had significantly increased odds of achieving a better clinical outcome (OR 3.33; 95% CI, 1.17 -9.52; P=0.026) (Table 1).

### Mechanical Thrombectomy

A mechanical thrombectomy procedure was carried out in acute ischemic stroke patients with large vessel occlusion, with or without previous intravenous thrombolysis before hospital admission. The selected time parameters after stroke inception to interventional procedures were reported in minutes (Table 1). The three-month modified Rankin Scale scores did not differ with different mechanical thromboembolism extraction devices (stent retrievers or aspiration devices) (P=0.98) (FIG. 12A). Intravenous thrombolysis was used prior to the mechanical interventional procedure in 101 (78%) patients, and intravenous thrombolysis was not used in 29 (22%) patients; these groups did not differ in terms of the three-month modified Rankin Scale scores for clinical outcome (P=0.459) (FIG. 12B). Treatment resulted in a Treatment in Cerebral Ischemia (TICI) score of 2-3 for recanalization in 115 (89%) of patients, while a TICI score of 0-1 was found in 14 (11%) patients, and these groups differed significantly in terms of the three-month modified Rankin Scale scores (P=0.027) (FIG. 12C) (Table 2). Patient overall mortality rate was 21% (Table 2).

TABLE 2
Thrombectomy procedure and clinical outcome.
Procedure description

| Variable | Categories | n (%) | OR (95% CI)* | P* |
|---|---|---|---|---|
| Intravenous thrombolysis (IIVT) | No | 29 (22) | 0.70 (0.28-1.75) | 0.497 |
| | Yes | 101 (78) | | |
| Alberta Stroke Program for Early | 0-5 | 1 (1) | NA | NA |
| CT Score (ASPECTS) | | | | |
| | 6-10 | 129 (99) | | |
| Alberta collateral scoring | 0-2 | 40 (31) | 0.17 (0.06-0.46) | <0.001 |
| | 3-5 | 89 (69) | | |
| SICH | No | 122 (97) | NA | NA |
| | Yes | 4 (3) | | |
| Non-SICH | No | 103 (82) | 1.81 (0.66-4.99) | 0.321 |
| | Yes | 23 (18) | | |
| Decompression | No | 125 (99) | NA | NA |
| | Yes | 1 (1) | | |
| Type of used anesthesia | Local anesthesia | 10 (7) | | |
| | IV sedation | 69 (51) | 0.70 (0.32-1.57) | 0.388 |
| | General anesthesia | 52 (39) | | |
| Previous statin medication | No | 50 (40) | 0.53 (0.24-1.17) | 0.166 |
| | Yes | 76 (60) | | |
| Previous antithrombotic medication | No | 57 (45) | 2.39 (1.09-5.26) | 0.032 |
| | Yes | 69 (55) | | |

Clinical outcome

| Variable | Categories | n (%) |
|---|---|---|
| The thrombolysis in cerebral | (0) - no perfusion | 8 (6) |
| infarction grading system | | |
| Treatment in Cerebral | (1) - minimal perfusion | 6 (5) |
| Ischemia (TICI) score | (2) - partial perfusion | 36 (28) |
| | (3) - complete perfusion | 79 (61) |
| NIH Stroke Scale at | Minor | 2 (2) |
| the time of arrival to | Moderate | 41 (33) |
| the hospital (NIHSS-entry) | Moderate to severe | 53 (42) |
| | Severe | 30 (23) |
| NIH Stroke Scale 24 hours | Minor | 28 (23) |
| after the mechanical | Moderate | 66 (54) |
| thrombectomy procedure | Moderate to severe | 18 (15) |
| (NIHSS-24 h) | Severe | 11 (8) |
| NIH Stroke Scale 7 days | Minor | 43 (36) |
| after the mechanical | Moderate | 57 (48) |
| thrombectomy procedure | Moderate to severe | 12 (10) |
| (NIHSS-7 d) | Severe | 7 (6) |
| Modified Rankin Scale 3 | 0-2 | 49 (47) |
| months after the mechanical | 3 | 17 (16) |
| thrombectomy procedure | 4-5 | 17 (16) |
| (three-month modified Rankin Scale) | 6 | 22 (21) |

^{*}Odds ratios for achieving better clinical status were computed (where possible), together with 95% confidence intervals and p-values of Chi-square test of independence (or Fisher’s exact test where necessary).

### Blood Coagulation

Hemocoagulation and the associated inflammation response analysis data are shown in Tables 3-6. Patients with a National Institutes of Health Stroke Scale (NIHSS) score >15 had significantly increased levels of vWF antigen (%) (P=0.003) on hospital admission for acute stroke therapy (median: 216; IDR: 137-374); patients with an NIHSS ≤15 had significantly lower vWF levels (median: 175; IDR: 132-276). Also, patients with lower three-month modified Rankin Scale scores of 3-6 had significantly increased levels of vWF (P<0.001) (median: 225; IDR: 160-379) compared with vWF levels in patients with three-month modified Rankin Scale scores of 0-2 (median: 174; IDR: 118-298) (Table 4).

TABLE 3
Hemocoagulation response and entry National
Institutes of Health Stroke Scale (NIHSS).

| | Status before the procedure | | |
|---|---|---|---|
| Variables | NIHSS ≤15 | NIHSS >15 | P* |
| PLT (Platelet) | 196 (124; 313) | 201 (151; 272) | 0.948 |
| PTC (Plateletcrit) | 0.2 (0.2; 0.3) | 0.2 (0.2; 0.3) | 0.359 |
| MPV (Mean Platelet Volume) | 11.7 (10.0; 13.0) | 11.2 (10.2; 12.6) | 0.309 |
| PDW (Platelet Distribution Width) | 14.1 (11.3; 17.8) | 13.8 (11.6; 17.2) | 0.731 |
| IPF (Immature Platelet Fraction) | 3.3 (1.7; 7.7) | 3.7 (1.6; 8.4) | 0.862 |
| P-LCR (Platelet Large Cell Ratio) | 38.8 (24.1; 47.2) | 34.0 (25.5; 46.0) | 0.321 |
| WBC (White Blood Cells) | 10.0 (6.4; 12.5) | 9.1 (6.1; 14.7) | 0.531 |
| Mono (Monocytes) | 0.6 (0.3; 1.1) | 0.6 (0.4; 0.9) | 0.708 |
| Hgb (Hemoglobin) | 128 (109; 151) | 133 (107; 149) | 0.545 |
| HTC (Hematocrit) | 0.383 (0.322; 0.443) | 0.390 (0.326; 0.450) | 0.397 |
| PT-R (Prothrombin Time-Ratio) | 1.1 (1.0; 1.3) | 1.1 (1.0; 1.3) | 0.981 |
| APTT-R (Activated Partial | 1.0 (0.8; 3.2) | 0.9 (0.8; 1.9) | 0.222 |
| Thromboplastin Time-Ratio) | | | |
| TT (Thrombin Time) | 18 (14; 30) | 18 (14; 30) | 0.974 |
| Fbg (Fibrinogen) | 2.42 (1.44; 3.47) | 2.37 (1.28; 3.70) | 0.651 |
| D-dimers | 3.3 (0.7; 15.1) | 3.4 (0.8; 24.8) | 0.596 |
| vWF (von Willebrand Factor) | 175 (132; 276) | 216 (137; 374) | 0.003 |
| FVIII (Factor VIII) | 174 (73; 280) | 177 (70; 363) | 0.397 |
| ADAMTS13 | 74 (21; 100) | 71 (16; 98) | 0.601 |
| vWF:ADAMTS13 ratio | 2.5 (1.5; 7.9) | 3.0 (1.9; 15.9) | 0.038 |
| CRP (C-Reactive Protein) | 4.1 (1.0; 13.8) | 5.2 (1.1; 31.6) | 0.122 |

*P-values of Mann-Whitney U test were computed to compare patients with different outcomes.
National Institutes of Health Stroke Scale (NIHSS).

TABLE 4
Hemocoagulation response and three-month modified Rankin Scale.

| | Outcome after 3 months | | |
|---|---|---|---|
| Variables | mRS 0-2 (n = 49) | mRS 3-6 (n = 56) | P* |
| PLT (Platelet) | 195 (147; 284) | 205 (148; 271) | 0.584 |
| PTC (Plateletcrit) | 0.24 (0.19; 0.33) | 0.23 (0.17; 0.31) | 0.987 |
| MPV (Mean Platelet Volume) | 11.6 (10.2; 12.7) | 11.2 (10.2; 12.8) | 0.529 |
| PDW (Platelet Distribution Width) | 14.1 (11.8; 17.6) | 13.8 (11.6; 17.6) | 0.497 |
| IPF (Immature Platelet Fraction) | 3.8 (1.6; 9.0) | 3.5 (1.8; 7.8) | 0.590 |
| P-LCR (Platelet Large Cell Ratio) | 37.4 (26.1; 46.4) | 34.0 (26.2; 47.1) | 0.493 |
| WBC (White Blood Cells) | 9.7 (6.1; 13.3) | 8.6 (6.1; 16.1) | 0.322 |
| Mono (Monocytes) | 0.6 (0.4; 1.2) | 0.6 (0.4; 1.0) | 0.333 |
| Hgb (Hemoglobin) | 130 (112; 152) | 130 (104; 152) | 0.489 |
| HTC (Hematocrit) | 0.386 (0.326; 0.446) | 0.390 (0.323; 0.452) | 0.987 |
| PT-R (Prothrombin Time-Ratio) | 1.07 (0.98; 1.36) | 1.10 (1.00; 1.38) | 0.430 |
| APTT-R (Activated Partial | 1.0 (0.8; 3.1) | 1.0 (0.8; 2.1) | 0.365 |
| Thromboplastin Time-Ratio) | | | |
| TT (Thrombin Time) | 18 (14; 32) | 19 (14; 30) | 0.912 |
| Fbg (Fibrinogen) | 2.4 (1.3; 3.5) | 2.2 (1.2; 3.8) | 0.566 |
| D-dimers | 2.6 (0.7; 10.7) | 4.4 (0.8; 26.0) | 0.029 |
| vWF (von Willebrand Factor) | 174 (118; 298) | 225 (160; 379) | <0.001 |
| FVIII (Factor VIII) | 150 (50; 320) | 190 (70; 340) | 0.267 |
| ADAMTS13 | 66 (15; 93) | 78 (20; 101) | 0.047 |
| vWF:ADAMTS13 ratio | 3.1 (1.6; 15.4) | 3.0 (1.7; 15.4) | 0.689 |
| CRP (C-Reactive Protein) | 4 (1; 24) | 5 (2; 31) | 0.078 |

*P-values of Mann-Whitney U test were computed to compare patients with different outcomes.

In the evaluation of vWF levels, patient blood type was also considered. In the patient study population, significantly lower vWF levels (P=0.007) were observed in patients with the O-blood type (median: 178: IDR: 117-263) compared with patients with other blood types (median: 207; IDR: 145-348). No significant difference (P=0.362) vWF levels were found between patients with a TICI score of 0-1 (median: 197; IDR: 151-411) and patients with a TICI score of score of 2-3 (median: 198; IDR: 135-326). There was a slight, but non-significant, increase in vWF levels in patients older than 65 years (median: 203; IDR: 137-325) compared with patients younger than 65 years (median: 181: IDR: 131-342) (P=0.153). Also, no significant difference in vWF levels (P=0.62) was found between patients with arrhythmia median: 199; IDR: 145-303) when compared with patients without arrhythmia (median: 198; IDR: 131-374). The Spearman's correlation coefficient between vWF levels and procedure duration was not significant (r=0.02). An increased systemic inflammatory response did not explain the increased vWF levels in patients with a poorer clinical outcome, as C-reactive protein (CRP) levels were not significantly different (Tables 3 and 4).

Significantly increased levels of D-dimers (P=0.029) were observed in patients with three-month modified Rankin Scale scores of 3-6 (median: 4.4; IDR: 0.8-26.0) compared with patients with three-month modified Rankin Scale scores of 0-2 (median: 2.6; IDR: 0.7-10.7) (Table 4). Patients older than 65 years had significantly higher D-dimer levels (P=0.022; median: 4.0; IDR: 0.9-25.1) than did younger patients (median: 2.6; IDR: 0.7-9.6). Patients with arrhythmia (n=51) had slightly diminished D-dimer values (median: 2.4; IDR: 0.8-19.3) than did those without arrhythmia (n=75; median 3.5; IDR: 0.8-19.2) (P=0.18), possibly because some patients with atrial fibrillation (AF) were on warfarin therapy.

There was no significant difference in ADAMTS13 levels between patients with an NIHSS score >15 and patients with an NIHSS score ≤15 (P=0.601) (Table 3). No significant difference in ADAMTS13 levels was found in patients with TICI scores of 0-1 when compared with patients with TICI scores of 2-3 (P=0.258). However, patients with three-month modified Rankin Scale scores of 3-6 had significantly increased levels of ADAMTS13 (P=0.047) (median; 78; IDR: 20-101) compared with patients with three-month modified Rankin Scale scores of 0-2 (median: 66; IDR: 15-93) (Table 4). Also, patients with severe neurological deficit, who has an NIHSS >15 before starting the mechanical interventional procedure had significantly increased vWF: ADAMTS13 ratios (P=0.038) (median: 3.0; IDR: 1.9-15.9) compared with the vWF: ADAMTS13 ratio in patients who has an NIHSS ≤15 (median: 2.5; IDR: 1.5-7.9) (Table 3).

### Inflammatory Response

Ischemia caused vascular thrombosis or thromboembolism results in serious homeostatic disruption of the affected tissue that results in an immune response. Following removal of the thrombus from the cerebral arteries supplying the brain, the total WBC count decreased slightly from the time of the procedure (median: 9.1; IDR: 6-13.0) to 2-5 days after mechanical removal (median: 8.8; IDR: 6.2-12.9) because of a significant decline in neutrophil count (P<0.001) from median 6.8 (IDR: 4.4-11.0) to median 5.8 (IDR: 3.9-9.9). The cell counts of monocytes, which are antigen-presenting cells, and their activated forms progressively increased, and cell counts of lymphocytes and eosinophils modulating the adaptive and immediate hypersensitivity reaction were also increased (Table 6).

Patients with a TICI score of 2-3 had an improved outcome, and had a significantly lower neutrophil count (P=0.006) (median: 5.6; IDR: 3.8-9.3) 5±2 days after the procedure, compared with patients with a TICI score of 0 -1 (median: 8.3; IDR: 5.1-11.0 (Table 6). This outcome corresponded to a significantly increased neutrophil count (P=0.002) in patients with NIHSS >15, 5±2 days after the procedure (median: 9.1; IDR: 4.6-11.0) compared with patients with NIHSS ≤15 (median: 5.7; IDR: 3.8-9M). The cell counts of total T-cells, helper T-cells, and cytotoxic T-cells were significantly lower (P=0.005, P=0.013; P=0.041, respectively) in patients with NIHSS >15 compared with those with NIHSS ≤15. The same trend was observed for cell counts of total T-cells and their subpopulation of helper T-cells between patients with three-month modified Rankin Scale scores of 3-6 and three-month modified Rankin Scale scores of 0-2 (P=0.006 and P=0.006, respectively) (Table 5).

TABLE 5
Inflammatory response and clinical outcome.
Inflammatory response 3-7 days after stroke

| | Outcome 7 days after stroke | | |
|---|---|---|---|
| Variable | NIHSS ≤15 | NIHSS >15 | P* |
| WBC | 8.5 (6.0-12.7) | 11.6 (6.6-14.9) | 0.019 |
| Neutrophils | 5.7 (3.8-9.0) | 9.1 (4.6-11.0) | 0.002 |
| Lymphocytes | 1.67 (1.02-2.34) | 1.19 (0.90-2.08) | 0.052 |
| Monocytes | 0.82 (0.52-1.25) | 0.88 (0.53-1.26) | 0.622 |
| Eosinophils | 0.20 (0.04-0.42) | 0.13 (0.02-0.31) | 0.136 |
| Basophils | 0.04 (0.02-0.08) | 0.03 (0.02-0.07) | 0.226 |
| T-cells | 1.16 (0.66-1.61) | 0.86 (0.59-1.25) | 0.005 |
| Helper T-cells | 0.77 (0.45-1.14) | 0.55 (0.38-0.92) | 0.013 |
| Cytotoxic T-cells | 0.37 (0.18-0.68) | 0.28 (0.16-0.39) | 0.041 |
| Natural killer | 0.24 (0.12-0.41) | 0.20 (0.08-0.46) | 0.264 |
| cells | | | |
| DR+ T-cells | 0.075 (0.032-0.170) | 0.054 (0.037-0.145) | 0.218 |
| B-cells | 0.19 (0.08-0.38) | 0.19 (0.08-0.44) | 0.595 |
| CD14+ cells | 0.68 (0.39-1.13) | 0.83 (0.45-1.17) | 0.308 |
| DR+ monocytes | 0.63 (0.37-0.97) | 0.62 (0.41-0.94) | 0.976 |
| CD16+ | 0.087 (0.041-0.159) | 0.107 (0.044-0.161) | 0.286 |
| monocytes | | | |
| Calprotectin | 6.0 (2.9-10.7) | 5.7 (3.9-12.7) | 0.383 |

Inflammatory response 3-7 days after stroke

| | Outcome after 3 months | | |
|---|---|---|---|
| Variable | mRS 0-2 (n = 49) | mRS 3-6 (n = 56) | P* |
| WBC | 8.1 (5.8-11.4) | 9.6 (6.6-13.7) | 0.061 |
| Neutrophils | 5.3 (3.1-7.9) | 6.8 (4.1-10.7) | 0.002 |
| Lymphocytes | 1.73 (1.29-2.78) | 1.47 (0.88-2.28) | 0.013 |
| Monocytes | 0.81 (0.48-1.18) | 0.88 (0.57-1.25) | 0.345 |
| Eosinophils | 0.20 (0.03-0.42) | 0.18 (0.04-0.35) | 0.231 |
| Basophils | 0.04 (0.02-0.08) | 0.04 (0.02-0.08) | 0.487 |
| T-cells | 1.27 (0.90-1.94) | 1.08 (0.64-1.56) | 0.006 |
| Helper T-cells | 0.81 (0.59-1.23) | 0.73 (0.36-1.06) | 0.006 |
| Cytotoxic T-cells | 0.38 (0.19-0.82) | 0.33 (0.16-0.62) | 0.090 |
| Natural killer | 0.25 (0.13-0.41) | 0.20 (0.10-0.38) | 0.127 |
| cells | | | |
| DR+ T-cells | 0.09 (0.04-0.19) | 0.07 (0.03-0.17) | 0.127 |
| B-cells | 0.19 (0.11-0.47) | 0.18 (0.06-0.38) | 0.243 |
| CD14+ cells | 0.66 (0.37-0.96) | 0.77 (0.48-1.17) | 0.114 |
| DR+ monocytes | 0.62 (0.35-0.92) | 0.65 (0.43-0.97) | 0.259 |
| CD16+ | 0.086 (0.036-0.198) | 0.103 (0.043-0.160) | 0.311 |
| monocytes | | | |
| Calprotectin | 5.8 (2.5-10.9) | 6.0 (4.3-12.6) | 0.066 |

*P-values of the Mann-Whitney U test were computed to compare patients with different outcomes.
National Institutes of Health Stroke Scale (NIHSS).

TABLE 6
Inflammatory responses.
Inflammatory response

| | Time of measurement | | |
|---|---|---|---|
| Variable | Entry | After 3-7 days | P* |
| WBC | 9.1 (6.1-13.0) | 8.8 (6.2-12.9) | 0.113 |
| Neutrophils | 6.8 (4.4-11.0) | 5.8 (3.9-9.9) | <0.001 |
| Lymphocytes | 1.23 (0.72-2.16) | 1.67 (1.01-2.57) | <0.001 |
| Monocytes | 0.58 (0.35-0.96) | 0.83 (0.52-1.27) | <0.001 |
| Eosinophils | 0.1 (0.0-0.2) | 0.2 (0.0-0.4) | <0.001 |
| Basophils | 0.04 (0.02-0.07) | 0.04 (0.02-0.08) | 0.654 |
| T-cells | 0.82 (0.43-1.50) | 1.15 (0.66-1.70) | <0.001 |
| Helper T-cells | 0.50 (0.27-1.01) | 0.75 (0.42-1.15) | <0.001 |
| Cytotoxic T-cells | 0.26 (0.11-0.54) | 0.35 (0.18-0.70) | 0.003 |
| Natural killer | 0.19 (0.0-90.46) | 0.23 (0.12-0.42) | 0.218 |
| cells | | | |
| DR+ T-cells | 0.061 (0.027-0.138) | 0.072 (0.033-0.173) | 0.052 |
| B-cells | 0.14 (0.06-0.29) | 0.19 (0.08-0.42) | <0.001 |
| CD14+ cells | 0.48 (0.28-0.80) | 0.71 (0.41-1.17) | <0.001 |
| DR+ monocytes | 0.45 (0.27-0.76) | 0.62 (0.38-0.95) | <0.001 |
| CD16+ | 0.039 (0.016-0.091) | 0.093 (0.039-0.162) | <0.001 |
| monocytes | | | |
| Calprotectin | 4.9 (2.6-9.8) | 5.8 (2.9-11.1) | 0.043 |

Inflammatory response 37 days after stroke

| | Treatment in Cerebral Ischemia (TICI) score | | |
|---|---|---|---|
| Variable | 0-1 | 2-3 | P** |
| WBC | 11.2 (7.3-13.7) | 8.5 (6.0-12.8) | 0.019 |
| Neutrophils | 8.3 (5.1-11.0) | 5.6 (3.8-9.3) | 0.006 |
| Lymphocytes | 1.2 (1.0-2.9) | 1.7 (1.0-2.5) | 0.307 |
| Monocytes | 0.90 (0.65-1.27) | 0.82 (0.50-1.26) | 0.426 |
| Eosinophils | 0.11 (0.03-0.27) | 0.19 (0.04-0.42) | 0.289 |
| Basophils | 0.04 (0.02-0.07) | 0.04 (0.02-0.08) | 0.454 |
| T-cells | 0.92 (0.60-1.96) | 1.15 (0.70-1.68) | 0.244 |
| Helper T-cells | 0.67 (0.39-1.14) | 0.75 (0.43-1.15) | 0.478 |
| Cytotoxic T-cells | 0.23 (0.18-0.83) | 0.37 (0.18-0.68) | 0.095 |
| Natural killer | 0.25 (0.06-0.42) | 0.22 (0.12-0.42) | 0.908 |
| cells | | | |
| DR+ T-cells | 0.049 (0.040-0.266) | 0.074 (0.032-0.172) | 0.490 |
| B-cells | 0.20 (0.13-0.38) | 0.19 (0.08-0.42) | 0.631 |
| CD14+ cells | 0.80 (0.55-1.18) | 0.69 (0.40-1.16) | 0.289 |
| DR+ monocytes | 0.60 (0.46-0.93) | 0.63 (0.37-0.95) | 0.694 |
| CD16+ | 0.114 (0.055-0.180) | 0.087 (0.039-0.162) | 0.242 |
| monocytes | | | |
| Calprotectin | 6.4 (3.4-11.3) | 5.8 (2.7-11.0) | 0.387 |

*P-values of the paired sign test were computed to compare the inflammatory response at different time points;
**P-values of the Mann-Whitney U test were computed to compare patients with different outcome.

### Histopathology of the Thrombus

Recanalization of the cerebral artery was successful in 115/131 patients (89%) from whom, 90 samples of thrombus were extracted, fixed, sectioned and analyzed histologically by tight microscopy (FIG. 13). From these samples of thrombus, it was possible to analyze five different areas in 79 samples via Carstairs' histochemical staining technique for platelets, which involved measuring the area of positive staining digitally on the image displayed on the monitor. The same method was used to analyze 84 samples (with more than three measurements from a single sample) for the immunostaining for the CD31 marker and 85 samples were analyzed immunohistochemically for the vWF marker. The analysis of the cellular composition of the extracted thrombi was done with the data from the use of intravenous thrombolysis administration prior to thrombectomy. TICI recanalization output, three-month modified Rankin Scale scores outcome, and plasma vWF measurements.

A large number of neutrophils were present in the surface layer of the extracted arterial thrombus (FIG. 13). These thromboemboli also had an increased area of vWF-positive immunostaining (FIG. 13). A significantly increased number of platelets were present in the mechanically extracted thrombi in patients with t TICI score of 2-3 compared with those with a TICI score of 0-1 (P=0.003). Thrombus samples from patients previously treated with intravenous thrombolysis had less fibrin (P=0.04) and fewer CD31-positive cells (P=0.056), although the reduction in CO31-positive immunostaining was not statistically significant.

Spearman's rank correlation coefficients showed a significant relationship between plasma vWF and the vWF found in the thromboembolus platelets (r=0.32), platelets (r=0.24), or fibrin (r=0.26). Also, in the thromboembolus structure, the area of immunostained vWF correlated with platelet count (r=0.53), CD31-positive cells (r=0.38), and fibrin (r=0.48), as did the amount of all CD31-positive cells with the number of neutrophils in the thrombus (r=0.68) (FIG. 14A). Spearman's rank correlation coefficient showed a significantly positive relationship between D-dimer levels and plasma vWF levels (r=0.21), well as three-month modified Rankin Scale scores (r=0.23) (FIG. 14A). Also, there were significant correlations between blood neutrophil levels and vWF levels within the thrombus (r=0.24), the numbers of natural killer (NK) cells and the fibrin content of the thrombus (r=0.26), and the numbers of lymphocytes and the fibrin content of the thrombus (r=0.24) (FIG. 14B).

### Conclusions

In cerebral artery thromboembolic occlusion leading to acute ischemic stroke, mechanical thrombectomy resulted in an increased Treatment in Cerebral Ischemia (TICI) score of 2-3 for the outcome of vessel recanalization in 89% of patients, and good clinical outcome was shown by a three-month modified Rankin Scale score of 0-2. In 47% of cases, Patients with worse clinical outcome had significantly increased levels of von Willebrand Factor (vWF) and the vWF: ADAMTS13 ratio was significantly increased in patients with a worse outcome at the time of onset of ischemic stroke. Increased plasma levels of vWF were associated with vWF-rich thrombus from cerebral arteries of patients with stroke, which were also platelet-rich, fibrin-rich, and neutrophil-rich. Patients treated with or without intravenous thrombolysis did not differ in terms of the three-month modified Rankin Scale score for clinical outcome.

These results demonstrate that ADAMTS13 metalloprotease can be used to treat a mammal having an embolism (e.g., a cerebral embolism) or at risk of developing an embolism (e.g., a cerebral embolism).

### Example 4

### Treating Brain Emboli

A human identified as having a cerebral embolism is administered a composition including one or more ADAMTS13 metalloproteases. In some cases, a physical examination (e.g., checking for blood pressure, atherosclerosis, and/or cholesterol crystals or clots in the blood vessels at the back of your eyes), blood tests (e.g., for clotting and/or blood sugar), imaging techniques (e.g., CT scanning such as CT angiography MRI such as magnetic resonance angiography or magnetic resonance venography, carotid ultrasound, X-ray imaging such as cerebral angiograph, electrocardiography (ECG or EKG) and/or echocardiogram) is used to identify the human as having a cerebral embolism.

The administered composition can reduce the severity of one or more symptoms of cerebral embolism e.g., slurring, confusion, difficulty understanding speech, paralysis, numbness, blurred vision, blackened vision, seeing double, headache, stumbling, dizziness, loss of balance, and/or loss of coordination) and/or can reduce the size and/or stickiness of one or more cardiac thromboemboli lodged in a cerebral artery.

### Other Embodiment

It is to be understood that while the invention has been described in conjunction with the detailed description thereof the foregoing description is intended to illustrate and not limit the scope of the invention, which is defined by the scope of the appended claims. Other aspects, advantages, and modifications are within the scope of the following claims.

What is claimed is:

1. A method for treating a cerebral embolism in a mammal, said method comprising:
administering a composition comprising an ADAMTS13 metalloprotease to a mammal identified as having an embolism, wherein said embolism comprises a cardiac thromboembolus in a cerebral artery;
wherein one or more symptoms of said embolism is reduced.

2. The method of claim 1, wherein said one or more symptoms are selected from the group consisting of slurring, confusion, difficulty understanding speech, paralysis, numbness, blurred vision, blackened vision, seeing double, headache, stumbling, dizziness, loss of balance, and loss of coordination.

3. A method for treating a cerebral embolism in a mammal, said method comprising:
administering a composition comprising an ADAMTS13 metalloprotease to a mammal identified as having an embolism, wherein said embolism comprises a cardiac thromboembolus in a cerebral artery, and wherein said cardiac thromboembolus is greater than 8 mm in size;
wherein said cardiac thromboembolus is reduced in size to less than 2 mm.

4. The method of claim 3, wherein said cardiac thromboembolus is reduced in size to less than 2 mm.

5. The method of claim 3, wherein said cardiac thromboembolus is eliminated.

6. A method for treating a cerebral embolism in a mammal, said method comprising:
administering a composition comprising an ADAMTS13 metalloprotease to a mammal identified as having an embolism, wherein said embolism comprises a cardiac thromboembolus in a cerebral artery;
wherein said cardiac thromboembolus is reduced in stickiness.

7. The method of claim 6, wherein said cardiac thromboembolus reduced in stickiness comprises a reduced amount of von Willebrand factor (vWF) or vWF multimers.

8. The method of claim 7, wherein said reduced amount of vWF or vWF multimers is at the thromboembolus surface.

9. The method of claim 8, wherein said vWF at the thromboembolus surface is present in one or more vWF/vWF multimers with platelets, fibrin, and inflammatory cells.

10. The method of claim 1, wherein said cerebral artery is selected from the group consisting of the internal carotid artery, anterior cerebral artery, the middle cerebral artery, the posterior cerebral artery, vertebral artery, and the basilar artery.

11. The method of claim 1, wherein said cardiac thromboembolus comprises one or more of red blood cells, fibrin, von Willebrand factor, vWF multimers, platelets, inflammatory cells, or plasma.

12. The method of claim 1, wherein said mammal is a human.

13. The method of claim 1, wherein said composition is administered by injection.

14. The method of claim 13, wherein said injection is an intra-arterial or intravenous injection.

15. The method of claim 14, wherein said intra-arterial injection is into one or more of the internal carotid artery, anterior cerebral artery, the middle cerebral artery, the vertebral artery, the basilar artery, and the posterior cerebral artery.

16. The method of claim 1, wherein said composition comprises about 100 Units to about 300 Units of ADAMTS13 metalloprotease per kg body weight of said mammal.

17. The method of claim 16, wherein said composition delivers about 200 Units of recombinant (rADAMTS13) metalloprotease per kg body weight of said mammal.

18. The method of claim 16, wherein said composition comprises about 12,000 Units of recombinant (rADAMTS13) metalloprotease.

19. The method of claim 1, wherein said method further comprising one or more antithrombotic treatments.

20. The method of claim 19, wherein said one or more antithrombotic treatments are selected from the group consisting of aspirin, warfarin, vitamin k antagonists, heparin, thrombin inhibitors, direct Xa inhibitors, streptokinase, urokinase, anistreplase, alteplase, reteplase, tenecteplase, staphylokinase, irreversible cyclooxygenase inhibitors, adenosine diphosphate receptor inhibitors, phosphodiesterase inhibitors, protease-activated receptor-1 antagonists, glycoprotein IIB/IIIA inhibitors, adenosine reuptake inhibitors, thromboxane inhibitors, mechanical thrombectomy, aspiration embolectomy, catheter-guided thrombolysis, percutaneous cerebral angioplasty, and stenting.
